(19) United States
(12) Patent Application Publication
(10) Pub. No.: US 2026/0314993 A1
(43) Pub. Date: Oct. 15, 2026

(54) COMPRESSION SLEEVE FOR QUANTITATIVE MEASUREMENTS

(71) Applicant: NOBLE INTERNATIONAL, LLC, Orlando, FL (US)

(72) Inventors: Jeffery A. LETTMAN, Orlando, FL (US); Randy CARDOZA, Orlando, FL (US); Jeff MILLER, Orlando, FL (US)

(21) Appl. No.: 19/104,536

(22) PCT Filed: Aug. 18, 2023
(86) PCT No.: PCT/US2023/030574

§ 371 Date: Feb. 18, 2025

Related U.S. Application Data
(60) Provisional application No. 63/399,062, filed on Aug. 18, 2022.

(51) Int. Cl. A61B 5/00; A61B 5/01; A61B 5/107; A61B 5/11; A61B 5/145; A61H 7/00

(52) U.S. Cl. CPC A61B 5/41; A61B 5/01; A61B 5/1075; A61B 5/11; A61B 5/14517; A61B 5/14546; A61B 5/4842; A61B 5/4878; A61B 5/6824; A61B 5/7271; A61B 5/7405; A61B 5/742; A61H 7/001; A61B 2560/0214; A61B 2560/0223; A61B 2560/0242; A61B 2562/0247; A61B 2562/0261; A61B 2562/0271; A61B 2562/164; A61H 2201/165; A61H 2201/5097; A61H 2205/06; A61H 2230/50

(57) ABSTRACT

Disclosed in embodiments herein is a wearable device and system for detecting data from a subject relating to a disease state, including a substrate for positioning around a target area of the subject, one or more sensors for detecting one or more symptoms of rheumatoid arthritis, comprising: joint swelling, temperature and/or mobility of the target area; and a power source.

### BACKGROUND

An important aspect of treating Rheumatoid arthritis (RA) is the ability to monitor disease activity based on a treat-to-target approach. Traditional disease activity measures for RA are unreliable. Inconsistencies in assessments made by physicians often occur, as well as an overestimation of improvement coupled with underestimation of disease progression in many cases. Traditionally, monitoring of disease activity in RA and gauging treatment responses have traditionally relied upon physician and patient-reported responses to composite tools, such as the Disease Activity Score (DAS-28), the Clinical Disease Activity Index, and the Simplified DAS. Unfortunately, these traditional measures for monitoring disease activity have been proven unreliable, thus a disadvantage to RA patients and treating healthcare professionals.

### SUMMARY

The ability to monitor disease activity in real-time based on identifiable symptoms, for RA patients these symptoms include swelling and/or decreased or restricted mobility in the joint or tender joints, can enhance treatment for patients. A wearable device that can detect disease symptoms and, optionally, correlate these symptoms with other data points and environmental factors as described herein is extremely effective in treating a rheumatic disorder, which may be caused by various factors. One such rheumatic disorder is RA.

In embodiments herein, a wearable device for providing compression to a target area of a subject may be provided. In non-limiting embodiments, the device may include an array of sensors and an ability to transfer data to and/or from the device for detection, analysis and tracking. The embodiments described herein provide a stream of data relating to factors for detecting disease state for RA as well as provide data points useful in determining best course of treatment. This quantifiable data gathered from the device provides more accurate datapoints relating to disease state and progression detection than self-reported metrics. The device compression also provides an increase in circulation to the target area, reducing pain.

The wearable device embodiments may include, in some examples, one or more sensors for detecting temperature, swelling, and other factors. Additionally, the device may include Near-field communication (NFC) technology to provide communication to and from, and energy to the device. The wearable device may be in communication with a smart device, including, for example a Smartphone for detecting and providing information relating to the device. For example, the smart device may log data including time, or location of the device, or various datapoints related to information detected by the sensor(s).

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which.

FIG. 1 shows an embodiment of the wearable device including a number of exemplary sensors positioned therein. the marker adhered to an injection site and an example of the AR content interacting with the marker in digital space. The wearable device detects and tracks the subject and marker using a sensor on the device.

FIG. 2 shows an embodiment of the wearable device and a smart device providing data transfer therebetween.

FIG. 3 shows an embodiment of the wearable device comprising a pocket for receiving a smart device, wherein the wearable device is worn on a user.

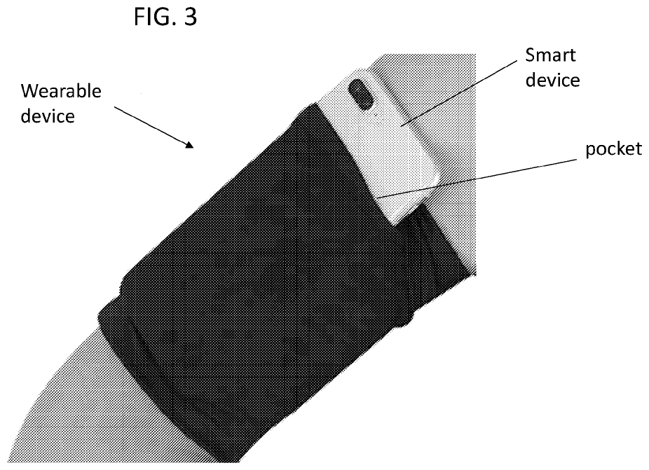

FIG. 4 shows an embodiment of the wearable device and exemplary sensors thereon.

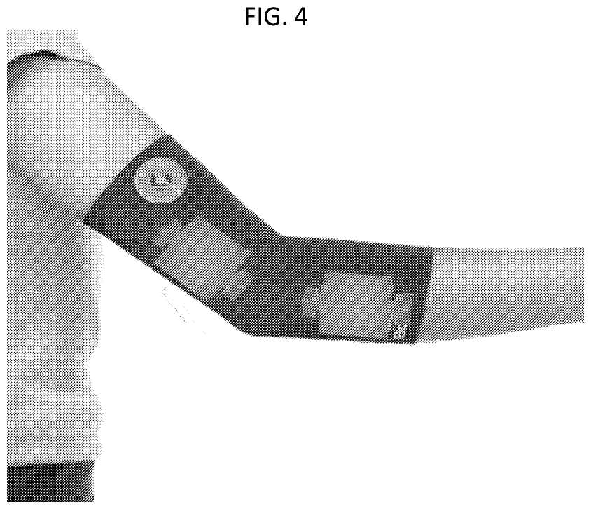

### DEFINITIONS

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” The modifier “about” used in connection with a quantity is inclusive of the stated value and has the meaning dictated by the context (e.g., includes the degree of error associated with measurement of the particular quantity). It is to be noted that all ranges disclosed within this specification are inclusive and are independently combinable.

The term “subject” as used herein refers to an individual. For example, the subject is a mammal, such as a primate, and, more specifically, a human. The term does not denote a particular age or sex. Thus, adult and newborn subjects, whether male or female, are intended to be covered. As used herein, patient or subject may be used interchangeably and can refer to a subject afflicted with a disease or disorder.

The terms “administering” or “administration” of an agent, drug, or peptide to a subject refers to any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

### DETAILED DESCRIPTION

Disclosed herein is a wearable device and system for gathering data and providing treatment to a subject. In some instances, the subject may present with symptoms of RA.

Embodiments described herein include devices and methods for detecting and/or monitoring the progression of a rheumatic disorder in a patient. The ability to monitor disease activity, optionally in real-time, based on identifiable symptoms which may include swelling, restricted movement, and/or tender joints can enhance treatment for patients. A wearable device or a system that includes a wearable device, that can detect disease symptoms and correlate these symptoms with other data points and environmental factors is extremely effective in treating a disease, such as RA, that can be caused by various factors.

In embodiments herein, a wearable device for providing compression to a target area of a subject may be provided. In non-limiting embodiments, the device may include an array of sensors and an ability to transfer data to and/or from the device for detection, analysis and tracking. The data may be transferred from the device to a smart device, whereby the smart device would provide analysis and tracking information based on the data provided. The embodiments described herein provide a stream of data relating to factors for detecting disease state for RA and other rheumatic disorders, as well as provide data points useful in determining the best course of treatment. This quantifiable data gathered from the device provides more accurate datapoints relating to disease state and progression detection than self-reported metrics. The device compression also provides an increase in circulation to the target area, reducing pain.

The wearable device embodiments may include, in some examples, one or more sensors for detecting temperature, swelling, and other factors. Additionally, the device may include Near-field communication (NFC) technology to provide communication to and from, and energy to the device. The wearable device may be in communication with a smart device, including, for example a Smartphone for detecting and providing information relating to the device. For example, the smart device may log data including time, or location of the device, or various datapoints related to information detected by the sensor(s).

Patients with a mean±SD joint temperature of 1.06±0.69 °F above central body temperature had a nearly 4-fold higher risk of new radiographic damage. Consequently, when a detection of a temperature at the target area meets these criteria, a determination of increased radiographic damage is made, which can inform medical practitioners to modulate patient exposure to radiation, such as by x-ray imaging or radiologic-therapies.

Presence of ACPA in established RA is associated with disease severity, while generation of ACPA at early developmental phases of RA can have a strong predictive value for progressing to the full-blown disease. Hence, development of ACPA may be of crucial importance to the pathogenesis of RA. Sensitivity (~40%) and specificity (over 95%) of ACPA as diagnostic biomarker are now recognized in early inflammatory arthritis patients with a suspicion of RA. ACPA is currently detected with a blood test. However, some forms of the enzymes responsible for citrullination, Peptidyl arginine deiminase (PAD), can be detected in epidermis, hair follicles, arrector pili muscles, and in sweat glands. This presents the possibility of detecting biomarkers associated with RA at skin level. Interestingly, anti-PAD1 were preferentially observed in patients with RA resulting in good discrimination between RA and controls from recciver operating characteristic (ROC) analysis.

In one embodiment, a smart device may be used to detect levels of a biomarker identified. For example, a camera component of a smart device may be used to take a photo to identify a level of biomarker in a sample. In examples wherein a wearable smart device is used, a photoresistor or photo reflector may be used to measure the intensity of reflected light. As an alternative to a colormetric readout, an assay can be used which may result in a measurable change in resistivity that can be measured by the device and used to create a treatment plan for the wearer.

Therapeutic drug monitoring (TDM) is the measurement of serum levels of a biologic drug with the aim of improving patient care. TDM can be combined with detection of antidrug antibodies that could neutralize the effect of the therapy. TDM provides reliable measurement of available drug in the serum and, if this is proven uncharacteristically low, to assess if antibodies towards the drug have developed. The three most commonly used approaches include an enzyme-linked immunosorbent assay (ELISA), a radioimmunoassay (RIA) and a homogeneous mobility shift assay (HMSA). ELISA uses a colormetric readout. Point of care testing with finger prick blood sampling can be used. In embodiments herein, microneedle patches may be used to expand this approach into wearable technologies. For example, a microneedle patch or other finger prick blood sample method may be used to identify the level of antibodies identified during RA treatment. This data may be used and interpreted to determine the best course of treatment for the patient. In some embodiments, this can be conducted in real time with a wearable device, and this data can be provided to a smart device, a user, or a third party.

Sensors used in detecting swelling in a joint as well as temperature changes may be a component of the wearable device 100. The data collection from the sensors and further analysis relating to the condition of the target area (i.e., the swollen joint area) may occur on the wearable device itself or on a smart device that is communicatingly connected to the wearable device. In one embodiment, all sensors for monitoring the user and the controller for receiving input from the sensors and providing an assessment relating to the data received may be provided entirely on the wearable device. In other embodiments, the sensors may be provided on the wearable device and the controller for receiving input from the sensors and/or the data collection and analysis components may be provided on a smart device that is in communication with the wearable device. In addition to sensors including contact sensors, fluid detection sensors, strain gauge sensors, temperature sensors, and the like for detecting swelling via the wearable device, imaging may also be used in detecting RA and related symptoms. Imaging may occur by way of simulated 3D scanning to detect and/or track swelling severity. Using continuous 2D imaging in combination with motion sensors may provide accurate 3D models to use for detection and treatment. In some embodiments, the motion sensors and/or the imaging component may be provided on a smart device that is in communication with the wearable device.

Detecting/analysis may occur within the wearable device, on the smart device, or a combination of both. The device, system and methods described herein in relation to detecting and treatment of RA may also be used for other conditions including, but not limited to Psoriatic Arthritis, Polyarticular juvenile idiopathic arthritis, Cryopyrin-Associated Periodic Syndromes, Systemic Lupus Erythematosus, and Periodic Fever Syndromes among others.

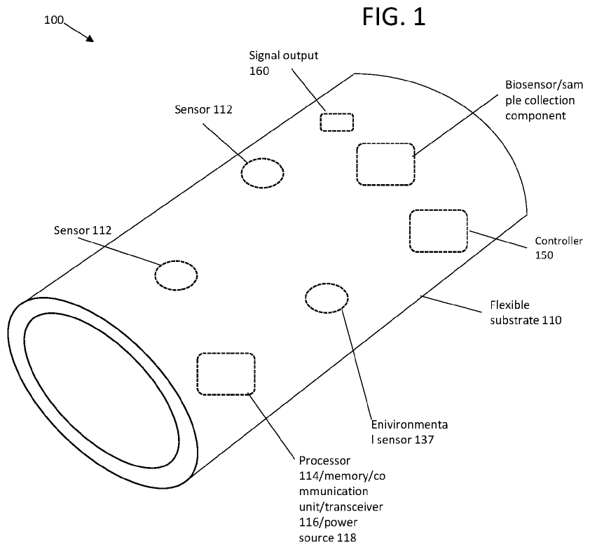

FIG. 1 provides a perspective view of a wearable device embodiment 100 for detecting data from a rheumatoid arthritis (RA) patient. The device 100 comprises a substrate 110 for positioning around a target area of the patient, one or more sensors 112 for detecting one or more factors of rheumatoid arthritis at the target area. The one or more sensors 112 may detect a resistance and/or a temperature at the target area as indicative of inflammation at the target area. The device may further include a controller 150 for receiving input from the one or more sensors 112, said controller 150 configured to measure the temperature and/or the resistance value, and/or to detect a change in the target area. The controller may include a baseline value received by input from a user or by monitoring and detecting data over a period of time, gathering a baseline for the user or wearer. Once a baseline has been input into the device or determined by the device over a period of time by monitoring and data gathering, a deviation from baseline may be detected. The deviation from baseline may be used in patient monitoring and treatment. Continuous monitoring of user data via the wearable device may provide data to determine patient adherence relating to treatment and/or medication, impacts of weather, exercise, and other factors. Monitoring of patients with the wearable device may provide a comprehensive, customized and effective treatment.¹ In some embodiments, the controller may receive pre-determined/baseline information and compare this data to new data points to detect improvement or decline of the condition .. The substrate 110 may include a flexible substrate configured to apply a pressure to and increase circulation to the target area during use of the device. Applying pressure to the target area helps to increase circulation in RA patients and/or decrease swelling in the target area, which serves to alleviate symptoms of RA including joint pain, swelling, and stiffness. ¹Catriona Grigor, Hilary Capell, Anne Stirling, Alex D McMahon, Peter Lock, Ramsay Vallance, Wilma Kincaid, Duncan Porter, “Effect of a treatment strategy of tight control for rheumatoid arthritis (t, he TICORA study): a single-blind randomized controlled trial.” PubMed, 2004 Jul 17-23; 364(9430): 263-9. doi: 10.1016/S0140-6736(04)16676-2. Accessed Jul. 18, 2022.

The one or more sensors 112 of the device 100 may include a strain gauge sensor, a pressure sensor, a light sensor, a contact sensor, a volumetric sensor, a circumference gauge sensor, and/or a thermal/temperature sensor. One or more of the sensors 112 may be used to detect a change in condition in or around the target area. The one or more sensors 112 may be used to detect swelling at the target area, for example, a strain gauge sensor may be used to detect swelling at or around the target area. A temperature sensor may also, or alternatively, be used to detect swelling at or near the target area.

The device 100 may include a power source 118. In some non-limiting embodiments, the one or more sensors 112 may include energy harvesting wireless sensors 112′, which may be powered by their environment via heat, light or motion, requiring no batteries to acquire and send data.

In an embodiment, the device 100 may include a power source 118 connected to the device by wired or wireless communication. The device 100 may include a conductive thread that may form part of the substrate 110. The conductive thread may form at least part of the substrate 110 and may include a conductive core including at least one conductive wire and a cover layer comprising flexible threads that covers the conductive core. The conductive core may be formed by twisting one or more flexible threads (e.g., silk threads, polyester threads, or cotton threads) with the conductive wire, or by wrapping flexible threads around the conductive wire. In one or more implementations, the conductive core is formed by braiding the conductive wire with flexible threads (e.g., silk). The cover layer may be formed by wrapping or braiding flexible threads around the conductive core. In one or more implementations, the conductive thread is implemented with a braided structure in which the conductive core is formed by braiding flexible threads with a conductive wire, and then braiding flexible threads around the braided conductive core to form at least part of the flexible substrate 110. The conductive wire may include a copper wire and may be insulated, in non-limiting embodiments. The conductive thread may interface with the power source 118, processor 114, sensors 112 and other components of the device 100.

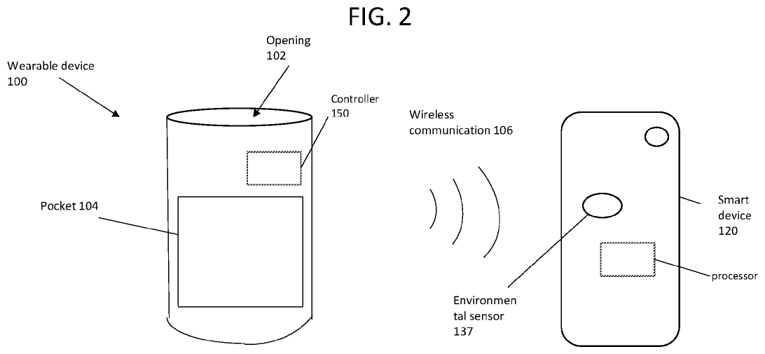

In some embodiments, the device 100 may include a wireless transceiver 116 configured to communicate to a smart device 120 as shown in FIG. 2. In some non-limiting embodiments, the device 100 may include an opening 102 for receiving a portion of a user, for example, a finger, an arm, a leg. Typically the sensors 112 of the device (shown in FIG. 1) are positioned on or around the joint area of the user when the device 100 is positioned at or near the target area. The device 100 may, in some non-limiting embodiments, include one or more pockets 104. The pockets 104 may be configured to receive a smart device 120 in one example. The smart device 120 may be wirelessly connected to the device 100 by way of wireless communication 106 as shown in the schematic of FIG. 2. In one example, the wireless communication may include near-field communication (NFC). Alternatively, the smart device 120 may be connected to the wearable device 100 by wired communication (not shown in FIG. 2).

In a further embodiment, the wearable device 100 may include a biosensor. The biosensor may be configured to receive a user sample to detect the presence of at least one biomarker for indication of disease. In one example, the disease may include RA. The at least one biomarker detected may include ACPA's (anti-citrullinated protein antibodies) to detect existence or non-existence of disease, and, optionally, disease severity. Disease severity may depend on the concentration of ACPA's detected in the user sample.

In one embodiment, the biosensor is associated with a processing circuit, said processing circuit configured to determine a concentration level of the at least one biomarker in the user sample. The biosensor may include or be associated with a sample receiving unit, the sample receiving unit may contact a surface of the user. The sample receiving unit may include, in one embodiment, a collection patch configured to receive a sample from the user. The sample may include a fluid sample comprising, for example, a fluid sample from a sweat gland or from the epidermis. In one embodiment, the collection patch may be in contact with a surface of the user, wherein microfluidic pathways may channel a sample obtained via the collection patch to a test chamber. The patch may be permanently or semi-permanently positioned in some instances. In other examples, the patch may be removable and replaceable.

In another embodiment, a system 200 for detecting, monitoring and treating rheumatoid arthritis (RA) in a subject is provided. The system 200 includes a wearable device 100 for detecting data from a subject, wherein the subject comprises rheumatoid arthritis (RA). The device may include a substrate 110 for positioning around a target area of the subject, and one or more sensors 112 for detecting one or more symptoms of rheumatoid arthritis, including: joint swelling, temperature and/or mobility of the target area. The system 200 may further include a power source 118, a wireless transceiver 116 configured to communicate data to a smart device 120, a processor, wherein the processor 114 optionally comprises baseline data for the subject. The processor 114 may compare the baseline data to data received from the one or more sensors 112 to detect a condition or a change in condition of the subject. In some examples, the condition may include RA. In some embodiments, the change of condition may include increased or decreased severity of RA, or an improvement or decline in RA disease state in the subject.

The system may include one or more environmental sensors 137 for detecting changes in the environment surrounding the device 100. The environmental sensors 137 may be provided on the smart device 120 communicatingly connected to the wearable device, or on or associated with the wearable device 100 itself. The one or more environmental sensors 137 comprise a humidity detection sensor including an air humidity sensor, a relative humidity sensor and/or an absolute humidity sensor, a temperature sensor, a light sensor, an air pressure sensor. The smart device 120 may be communicatingly connected to the wearable device by wired or wireless connection. The device 100 may be powered by the smart device 120 in one embodiment. The smart device 120 may comprise various components of the system 200, including, for example, the processor 114. The processor 114 may be configured to store and track data including RA factors, including, but not limited to diet of the patient, exercise of the patient, medication, medication administration, ambient weather, position of the wearable device on the user, location of the wearable device (by GPS), date, time, and history of each factor. The system may identify trends in data to assist in treatment of the disease. In particular, the system may provide information helpful in the treatment of RA, for example, where many factors may contribute to disease. In some embodiments, the device 100 or the system 200 may include a signal output component 160 for providing information to a user. Processing of the system may occur on the wearable device via the controller, on the smart device 120 via the smart device processing system, or both. Data may be communicated between the wearable device and the smart device, or from either the wearable device 100 or the smart device 120 to a third party or to a user.

The signal output component may include a visual, audible, olfactory, or other output to deliver information, including lights, an LCD screen, or a speaker, for example. In one embodiment of the system 200, the smart device 120 is configured to store and track data including RA factors, including diet, exercise, medication, medication administration, ambient weather, location of wearable device, date, time, and history of each factor. Other factors that may contribute to RA include sleep, stress, smoking, infections. Smoking increases pain and stiffness of individuals and can decrease the effectiveness of medications. Stress may worsen symptoms of RA.² Health issues associated with obesity including metabolic syndrome, for example, can exacerbate RA symptoms.³ Additionally, an infection's impact on the immune system may trigger RA.⁴ Dietary Interventions may help manage symptoms of RA in some cases.⁵ Some scientific evidence suggests that weather or seasonal changes may impact one's RA symptoms.⁶ ²https://www.ncbi.nlm.nih.gov/pmc/articles/PMC2911881/³https://www.ncbi.nlm.nih.gov/pmc/articles/PMC3557423/⁴https://www.ncbi.nlm.nih.gov/pmc/articles/PMC4131749/⁵https://www.frontiersin.org/articles/10.3389/fnut.2017.00052/full⁶https://www.ncbi.nlm.nih.gov/pmc/articles/PMC6339394/

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

What is claimed is:

1. A wearable device for detecting and/or monitoring the progression of a rheumatic disorder in a patient, comprising:
a substrate for positioning around a target arca of the patient;
one or more sensors for detecting one or more factors of the rheumatic disorder at the target area, wherein said one or more sensors detect mobility, swelling, temperature, or a combination thereof, as indicative of inflammation at the target area; and
a controller for receiving input from the one or more sensors, said controller configured to measure the temperature and/or the resistance value, and/or to detect a change in the target area and
a power source.

2. The wearable device of claim 1, wherein the substrate comprises a flexible substrate configured to apply pressure to and increase circulation to the target area.

3. The wearable device of claim 1, wherein the one or more sensors comprises a strain gauge sensor, a pressure sensor, a light sensor, a contact sensor, a volumetric sensor, a circumference gauge, an RTD temperature sensor, and/or a temperature sensor.

4. The wearable device of claim 1, wherein the one or more sensors comprise energy harvesting wireless sensors.

5. The wearable device of any of claims 1-4, wherein the power source comprises a conductive thread associated with the substrate for powering the device.

6. The wearable device of claim 3, wherein the strain gauge sensor is configured for detecting a swelling in the target area adjacent to the strain gauge sensor.

7. The wearable device of any of claims 1-6, wherein the power source is connected to the device by wired or wireless communication.

8. The wearable device of any of claims 1-7, further comprising a wireless transceiver configured to communicate to a smart device.

9. The wearable device of any of claims 1-8, wherein the device is communicatingly connected to a smart device via a wired or wireless connection.

10. The wearable device of any of claims 1-9, wherein said wearable device comprises a biosensor configured to receive a user sample to detect the presence of at least one biomarker for indication of disease.

11. The wearable device of claim 10, wherein the biosensor is associated with a processing circuit, said processing circuit configured to determine a concentration level of the at least one biomarker.

12. The wearable device of claims 10-11, wherein the at least one biomarker comprises ACPA's (anti-citrullinated protein antibodies) to detect existence or non-existence of disease, and, optionally, to detect disease severity.

13. The wearable device of claims 10-12, wherein the biosensor comprises a sample receiving unit on a surface of the device, wherein said sample receiving unit contacts a surface of the patient.

14. The wearable device of claim 13, wherein the sample receiving unit comprises a patch configured to receive a sample from a user.

15. The wearable device of claim 14, wherein the sample comprises a fluid sample comprising, for example, a fluid sample from a sweat gland or epidermis of the patient.

16. The wearable device of any of claims 1-10, further comprising a pocket for receiving a smart device.

17. The wearable device of claim 9, wherein the wireless connection comprises near-field communication (NFC).

18. The wearable device of any of claims 1-17, wherein the substrate applies pressure to a target area to decrease swelling in the target area.

19. The wearable device of claim 1, wherein the controller may receive baseline information data and compare this data to one or more new data points to detect improvement or decline of the condition, said baseline information data may be provided via a calibration routine upon powering on the device.

20. The wearable device of claim 1, wherein the controller may receive baseline information data and compare this data to one or more new data points collected over time during use of the device to detect improvement or decline of the condition, wherein the baseline information data may be determined over a period of time, whereby the device continuously monitors the target area in question and retroactively establishes baseline information data based on the data identified.

21. A system for detecting, monitoring and treating rheumatoid arthritis (RA) disease in a subject, comprising:
a wearable device for detecting data from a subject, wherein the subject comprises rheumatoid arthritis (RA), the device comprising a substrate for positioning around a target area of the subject, and one or more sensors for detecting one or more symptoms of rheumatoid arthritis, comprising: joint swelling, temperature and/or mobility of the target area;
a power source;
a wireless transceiver configured to communicate data to a smart device;
a processor, wherein the processor optionally comprises baseline data for the subject;
wherein the processor compares the baseline data to data received from the one or more sensors to detect a change in condition of the subject.

22. The system of claim 21, wherein the change in condition of the subject comprises an improvement or a decline in RA disease state, or alternatively, no change in RA disease in the subject.

23. The system of any of claims 21-22, wherein the one or more sensors comprises a strain gauge sensor, a pressure sensor, a light sensor, a contact sensor, and/or a temperature sensor.

24. The system of any of claims 21-23, further comprising one or more environmental sensors for detecting changes in the environment surrounding the device.

25. The system of claim 24, wherein the one or more environmental sensors comprise a humidity detection sensor, a fluid detection sensor, a moisture detection sensor, a temperature sensor, a light sensor, and/or an air pressure sensor.

26. The system of any of claims 21-25 further comprising a smart device, wherein the smart device is communicatingly connected to the wearable device.

27. The system of claim 26, wherein the wearable device is powered by the smart device.

28. The system of claim 26, wherein the smart device comprises the processor.

29. The system of claim 21, wherein the processor is configured to store and track data including RA disease factors, comprising: diet, exercise, medication, medication administration, ambient weather, and location of wearable device, date, time, and a history of each factor.

30. The system of any of claims 21-29, wherein the wearable device comprises signal output component for providing information to a user.

31. The system of claim 26, wherein the smart device is configured to store and track data including RA discasc factors, comprising: dict, exercise, medication, medication administration, ambient weather, location of wearable device, date, time, and history of each factor.

32. The wearable device of claim 9, wherein the controller may transmit data to the smart device, said smart device for comparing data to detect improvement or decline of the condition.

33. The wearable device of claim 32, wherein the smart device provides an output to a user or a third party to assess treatment guidelines for the user based on the data.
